(19) United States
(12) Patent Application Publication
(10) Pub. No.: US 2010/0062059 A1
(43) Pub. Date: Mar. 11, 2010

(54) S-Nitrosothiols Containing Composition for the Treatment of Fatty Liver Diseases, Obesity and Other Diseases Associated with the Metabolic Syndrome and the Use of Such Compositions

(75) Inventor: Marcelo Ganzarolli De Oliveira, Campinas (BR)

Correspondence Address: THE NATH LAW GROUP, 112 South West Street, Alexandria, VA, 22314, US

(73) Assignee: PROTAGEN AKTIENGESELLSCHAFT, DORTMUND (DE)

(21) Appl. No.: 12/226,406

(22) PCT Filed: Apr. 20, 2007
(86) PCT No.: PCT/BR2007/000099

§ 371 Date: Jan. 9, 2009

(30) Foreign Application Priority Data
Apr. 20, 2006 (BR) P10602397-5
Apr. 20, 2007 (BR) C10602397-5

(51) Int. Cl. A61K 33/00; A61K 31/197; A61K 31/401; A61K 38/38; A61K 38/16; A61K 31/355; A61K 31/4439; A61K 31/40; A61K 31/4406; A61K 9/28; A61K 9/48; A61P 1/16

(52) U.S. Cl. 424/463; 424/718; 514/562; 514/423; 514/12; 514/6; 514/458; 514/342; 514/356; 424/474

(57) ABSTRACT

The present invention refers to pharmaceutical compositions containing S-nitrosothiols as active principle. The referred compositions are intended for the treatment of the fatty liver disease and other diseases associated with the metabolic syndrome. The composition is administered either orally or rectally.

### FIELD OF THE INVENTION

The present invention refers to the development of pharmaceutical composites containing S-nitrosothiols as the active principle. The referred compositions are intended for the treatment of the fatty liver diseases by the oral or rectal route of administration to the patients. Additionally, the present invention refers to a method of treatment of the fatty liver diseases using the referred pharmaceutical composites.

### BACKGROUND OF THE INVENTION

The nonalcoholic fatty liver disease (NAFLD), also described as nonalcoholic steatohepatitis (NASH), encompasses a wide array of diseases, ranging from benign non-inflammatory steatosis up to fibrotic steatohepatitis and cirrhosis. NAFLD is recognized as an important chronic liver disease with clinical and histopathological characteristics very similar to those of the alcoholic form of the disease, however, without ethanol ingestion (Falck-Ytter, Y; Younossi, Z. M; Marchesini, G.; Mccullough, A. J, Clinical features and natural history of nonalcoholic steatosis syndromes, Seminars in liver disease, 2001; 21:17-26/Powell, E. E.; Cooksley, W. G.; Hanson, R.; Searle, J.; Halliday, J. W.; Powell, I. W. The natural history of nonalcoholic steatohepatitis: a follow-upstudy of forty-two patients for up to 21 years, Hepatology, 1990; 11:74-80).

NAFLD has been referred to as one of the most common types of hepatic disease and has been associated especially with the increasing prevalence of obesity. In the United States, NAFLD currently presents a high incidence of cases with epidemic characteristics (Zafrani, E. S., Non-alcoholic fatty liver disease: an emerging pathological spectrum. Virchows Arch, 2004; 444:3-12). Approximately half the patients with nonalcoholic steatohepatitis develop hepatic fibrosis, 15% develop cirrhosis and 3% may progress into hepatic failure or liver transplant (Marchesani, G.; Brizi, M; Morselli-Labate, A. M; Bianchi, G.; Bugianesi, E.; McCullough, A. J.; Forlani, G.; Melchionda, N., Association of Nonalcoholic Fatty Liver Disease with Insulin Resistance, American Journal of Medicine, 1999; 107(5): 450-455/Koteish, A.; Diehl, A. M, Animal Models of Steatosis, Seminars in Liver Disease, 2001; 21(1): 88-104).

Although several predisposing factors have been related to NAFLD, such as plurimetabolic syndrome (which includes obesity, type 2 diabetes mellitus, dyslipidemia, visceral adiposity and hypertension), jejunoileal bypass, protein-calorie malnutrition, prolonged parenteral nutrition or drug use, the pathogenesis and the therapeutics of this condition as well as the possibility of progression to chronic liver disease remain unclear. Among the main hypotheses that have been implicated in the physiopathogenesis of NAFLD, the following key factors stand out: insulin resistance as an initial condition (first hit) for the increased afflux of fatty acids to hepatocytes (Marchesini, G.; Brizi, M; Bianchi, G.; Tomassetti, S.; Bugianesi, E.; Lenzi, M; McCullough, A. J; Natale, S.; Forlani, G.; Melchionda, N., Nonalcoholic fatty liver: a feature of the metabolic syndrome, Diabetes, 2001; 50:1844-1850/Marchesini, G.; Bugianesi, E.; Forlani, G.; Cerrelli, F.; et al., Nonalcoholic fatty liver, steatohepatitis, and the metabolic syndrome, Hepatology, 2003; 37(4):917-923/Chituri, S.; Abeygunasekera, S.; Farrell, G. C. et al., NASH and insulin, resistance: insulin hypersecretion and specific association with the insulin resistance syndrome, Hepatology, 2001; 35(2):373-379/Festa, A.; D'Agostino, R. B.; Howard, G.; Mykkanen, L.; Tracy, R. P.; Haffner, S. M., Chronic subclinical inflammation as part of the insulin resistance syndrome. The Insulin Resistance Atherosclerosis Study (IRAS), Circulation, 2000; 201:42-47) and the increase of the oxidative stress as a triggering factor for inflammation and fibrosis (Chitturi, S.; Farrell, G., Etiopathogenesis of Nonalcoholic Steatohepatitis, Seminars in Liver Disease, 2001; 21(1):27-41/Leclercq, G. R. I.; Farrell, G. C., Nonalcoholic steatosis and steatohepatitis-II. Cytochrome P-450 enzymes and oxidative stress, Am J Physiology-gastrointestinal and Liver Phys, 2001; 281:1135-1139/Yang, S. Q.; Zhu, H.; Li, Y; Gabrielson, K; Trush, M. A.; Diehl, A. M., Mitochondrial Adaptations to Obesity-Related Oxidant Stress, Arch Biochem Biophysics, 2000; 378(2): 259-268).

Hyperinsulinemia favors lipogenesis and inhibits lipolysis, even in the liver, which increases the afflux of fatty acids to this organ. The excessive offer of fatty acids to the liver, as a consequence of disturbances in triglyceride exportation, may promote the depletion of mitochondrial oxidation and increase in the production of reactive oxygen species (ROS), as well as activation of other lipid oxidation pathways (peroxisomal and microsomal oxidation), which, in turn, triggers the production of more ROS and accentuates the hepatic oxidative stress. This increase is known to cause lipid peroxidation, whose intermediate products are important pro-inflammatory agents and seem to activate Ito cells, thus creating favorable conditions to fibrogenesis (Lee, K. S.; Buck, M; Houglum, K.; Chojkier, M., Activation of hepatic stellate cells by TGF alpha and collagen type I is mediated by oxidative stress through c-myb expression, J Clin Invest, 1995; 96:2461-2468/Curzio, M.; Esterbauer, H.; Dianzani, M. U., Chemotactic activity of hydroxyalkenals on rat neutrophils, Int J Tiss Reac, 1985; 7:137-142).

It is also known that the microvesicular steatosis is frequently associated to severe hepatic dysfunctions that may involve defects in the free fatty acid β-oxidation chain. Macrovesicular steatosis, in turn, results from a complex combination of pathogenic alterations that includes increased release, inadequate oxidation and decreased secretion of various forms of lipids in the liver and may cause necroinflammation and fibrosis (Brunt, E. M., Nonalcoholic Steatohepatitis: Definition and Pathology, Seminars in Liver Disease, 2001; 21(1):3-16).

The findings of previous experimental studies by Oliveira et al. demonstrated an increase in the hepatic oxidative stress in steatotic livers of Wistar rats induced by a choline-deficient diet (a diet that inhibits the exportation of triglycerides from the hepatocytes) (Oliveira, C. P. M. S.; Gayotto, L. C. C.; Tatai, C.; Della Nina, B. I; Janiszewski, M; Lima, E. S.; Abdalla, D. S. P.; Lopasso, F. P.; Laurindo, F. R. M; Laudanna, A. A., Oxidative stress in the pathogenesis of nonalcoholic fatty liver disease, in rats fed with a choline-deficient diet, J Cell Mol Med, 2002; 6(3):399-406) and a decrease in steatosis in choline-deficient diet fed Wistar rats treated with C vitamin (a classic antioxidant agent) using the same model of NAFLD (Oliveira, C. P.M. S.; Gayotto, L. C. C.; Tatai, C.; Della Nina, B. I.; Lima, E. S.; Abdalla, D. S. P.; Lopasso, F. P; Laurindo, F. R. M; Carrilho, F. J., Vitamin C and vitamin E in prevention of nonalcoholic fatty liver disease (NAFLD) in choline-deficient diet fed rats, Nutrition Journal, 2003; 2: 9). These findings and several other evidences indicate that NAFLD may actually be associated with oxidative stress and lipid peroxidation. Given that the oxidative stress involves mitochondrial β-oxidation of short-, middle- and long-chain fatty acids, with production of free electrons, hydrogen peroxide (H₂O₂) and oxygen-reactive species, glutathione and vitamin E (two other notorious antioxidant agents) may exert a therapeutic role in this disease (McCullough, A. J., Update on Nonalcoholic Fatty Liver Disease, Journal of Clinical Gastroenterology, 2002; 34(3): 255-262).

An important characteristic of these oxidative physiologic processes is that an increase in the levels of free fatty acids in the fatty liver may promote propagation of lipid peroxidation under conditions of oxidative stresses (Robertson, G.; Leclercq, I; Farrel, G. C., Nonalcoholic steatosis and steatohepatitis II. Cytochrome P-450 Enzymes and Oxidative Stress, American Journal of Physiology-Gastrointestinal and Liver Physiology, 2001; 281: G1135-G1138). Nevertheless, the oral administration of classic antioxidants for the treatment of NAFLD has not demonstrated remarkable therapeutic effects in the treatment of humans. In most cases, the beneficial effects are only discrete and do not inhibit the progression of these diseases. Other managements based on specific feeding regimens have also been shown to be palliative treatments. In spite of the evidences that oxidative stress is involved in the genesis and progression of NAFLD, there is no current therapeutic approach based on the administration of drugs by the oral route or other routes that can be considered as effective in the treatment of this disease.

In addition to the typical antioxidants, like those mentioned above, it is known that endogenously produced nitric oxide (NO) may act as a potent inhibitor of lipid peroxidation through the inhibition of peroxidation initiators, such as peroxidase enzymes, or the blockage of radicalar propagation reactions (Hubbo, H.; Darley-Usmar, V.; Freeman, B. A., Nitric Oxide Regulation of Tissue Free Radical Injury, Chemical Research in Toxicology, 1996; 9(5): 809-820).

The knowledge of this antioxidant action of nitric oxide followed the discovery, in 1987, that NO is the endothelium-derived relaxing factor (EDRF) and that this diatomic molecule is endogenously produced in the cells of mammalians by a family of enzymes called NO-sintases (NOSs). This discovery prompted the development of a large number of studies on the chemical and physiological properties of NO (Koshland Jr., D. E.; Culotta E., The Molecule of the Year. Science, 1992; 258:1861-1865/Ignarro, L. J.; Buga, G. M; Wood, K. S.; Byrns, R. E.; Chaudhuri, G., Endothelium-Derived Relaxing Factor Produced and Released from Artery and Vein is Nitric Oxide, Proc. Natl. Acad. Sci. U.S.A., 1987; 84(24): 8265-8268).

It is currently well accepted that NO plays essential regulatory roles as an intracellular and intercellular messenger and is one of the key species involved in the immune system response (Giustarini, D.; Milzani, A.; Colombo, R.; Dalle-Donne, I.; Rossi, R., Nitric Oxide and S-nitrosothiols in human blood, Clinica Chimica Acta, 2003; 330(1-2):85-98).

Several studies have demonstrated that failures in the endogenous production of NO may lead to the development of diseases associated with oxidative stress (Hogg, N.; Kalyanaraman, B., Nitric Oxide and Lipid Peroxidation, Biochimica et Biophysica Acta, 1999; 1411(2-3): 378-384/Violi, F.; Marino, R.; Milite, M. T.; Loffredo, L., Nitric Oxide and its Role in Lipid Peroxidation, Diabetes/Metabolism Research and Reviews, 1999; 15(4):283-288).

In the mammalians, including humans, endogenously synthesized NO is transported by peptides that contain the sulfhydryl functional group (R—SH) in the form of S-nitrosothiols (RSNOs), which, in turn, can transfer NO to endogenous receptor molecules in transnitrosation reactions. Two of the endogenous NO carriers that have already been identified in the literature are S-nitrosogluthatione and S-nitrosoalbumin.

There are currently no patent applications or information in the literature regarding the administration of S-nitrosothiols or any other oxide nitric donor in animal models of NAFLD or for the treatment of human patients diagnosed with this disease or other diseases associated with the metabolic syndrome, such as obesity, type 2 diabetes mellitus, dyslipidemia, visceral adiposity and hypertension. Nevertheless, there are patents from the period between 1996 and 2004, which mention the treatment of NASH with other therapeutic agents. Among these patents, the following stand out: US 2.004.105.870—This invention proposes methods and compositions for the treatment or attenuation of nonalcoholic hepatic steatosis and the, pharmaceutical formulations for its administration in human beings. The mentioned compositions include lecithin, antioxidants and B-complex vitamin to be administered by either the parenteral or the oral route as tablets or powder; U.S. Pat. No. 6.297.229—Proposes therapeutic methods for the treatment of nonalcoholic hepatic steatosis with effective amounts of ursodeoxycholic acid or a pharmaceutically acceptable derivate of this compound; U.S. Pat. No. 6.596.762—Refers to compositions that consist essentially in defined amounts of soluble E vitamin mixed with carotenoids and selenium, with possible addition of an additional unspecified agent for the treatment; U.S. Pat. No. 5.760.010—This invention is directed at the oral administration of erythromycin or a derivate of this drug for the treatment of human patients with liver diseases or disorders. The routes of administration may include the oral, intramuscular, subcutaneous, transdermical, intravenous or other common routes of drug administration to the patients. Additionally, this invention proposes the oral administration of clarithromycin, troleandomycin and azithromycin, for the treatment of human patients with liver diseases or disorders, including, but not limited to, nonalcoholic hepatic steatosis and Reye's syndrome; BR 0.311.843-6—This invention refers to the use of several types of gastric inhibitory polypeptide (GIP) receptor antagonists to attenuate the after-meal insulin response to GIP in animals and humans, in order to prevent, reduce, inhibit and/or treat nonalcoholic hepatic steatosis due to its potential for prevention and/or reversion of hyperinsulinemia and insulin resistance. This invention is based on the administration of an effective amount of an antagonistic agent, such as GIP antagonist or an antisense molecule, to antagonize, block, inhibit or withdrawal the glucose-dependent insulinotropic polypeptide (GIP) receptor.

The patent WO 9.416.740 mentions the use of S-nitrosothiols only for the treatment or prevention of hepatic diseases caused by alcohol ingestion or exposure to pharmacological agents or industrial toxins. To treat such diseases, this patent proposes the use of nitric oxide-releasing compounds, such as S-nitrosothiols, thionitrites, sydnonimines, furoxanes, organic nitrates, nitroprussiate, nitroglycerin, iron-nitrosyl compounds, or other related compounds. This patent also mentions that alcohol-induced hepatic disease would be prevented by the administration of a therapeutically effective amount of arginine or a nitric oxide-releasing compound together with the ingestion of alcoholic beverages. Nevertheless, this invention cited as a reference does neither explain nor even mention the application of S-nitrosothiols in the treatment of liver diseases not associated with alcohol ingestion or exposure to pharmacological or toxic agents. The patent WO 9.416.740 does not refer to the oral administration of S-nitrosothiols for the treatment of other diseases associated with the metabolic syndrome, such as obesity, type 2 diabetes mellitus, dyslipidemia, visceral adiposity and hypertension, as proposed in the resent invention. In addition, the patent WO 9.416.740 does not mention either the form of administration based on the previous mixture of a nitrosable thiol with sodium nitrite, in such a way that the S-nitrosothiol is generated immediately before its administration, or the other pharmaceutical composites described in the present invention.

The literature has shown that no classic antioxidant is capable of exerting the modifying actions of S-nitrosothiols because these actions are associated with the presence of the NO molecule in the S-nitrosothiols and with the capacity of these compounds of transferring NO to receptor molecules (e.g.: enzymes and other proteins) during transnitrosation reactions. This is a unique characteristic of the S-nitrosothiols, which allows classifying these compounds as nitrosating agents. The most common treatments with antioxidant substances or diets have been shown to be a palliative management in most cases.

As observed by the review of NAFLD history, it is of great interest that other drugs and pharmaceutical formulations using S-nitrosothiols are developed due to the major therapeutic potential of these compounds in the treatment of the nonalcoholic fatty liver disease and other diseases associated with the metabolic syndrome, such as obesity, type 2 diabetes mellitus, dyslipidemia, visceral adiposity and hypertension, by the oral route of administration.

### BRIEF DESCRIPTION OF THE INVENTION

The present invention refers to the development of pharmaceutical composites for the treatment of the Fatty Liver Diseases, more specifically, the Nonalcoholic Fatty Liver Disease (NAFLD), and other diseases associated with the metabolic syndrome, such as obesity, type 2 diabetes mellitus, dyslipidemia, visceral adiposity and hypertension. The pharmaceutical composites contain as the active principle S-nitrosothiols in their pure form as well as S-nitrosothiols produced from precursor thiols, immediately before their administration. Additionally, the present invention refers to the therapeutic use of the referred composites as a treatment method for liver diseases,. specifically NAFLD, and other diseases associated with the metabolic syndrome, such as obesity, type 2 diabetes mellitus, dyslipidemia, visceral adiposity and hypertension, which encompasses a wide array of diseases, ranging from benign non-inflammatory steatosis up to fibrotic steatohepatitis and cirrhosis. The referred treatment method comprehends the administration of the referred composites by the oral or rectal route. The mechanism of action of the active principle of the pharmaceutical composites of the present invention is based not only on the antioxidant action of S-nitrosothiols but also on its nitrosating action, which is capable of modifying the activity of enzymes involved in the synthesis and transport of lipids in the liver.

The main advantage of the present invention is that the method of treatment by the oral or rectal administration of the pharmaceutical composites might potentially prevent the development of the nonalcoholic fatty liver disease or even promote the regression of NAFLD and other diseases associated with the metabolic syndrome after their onset in the patient's organism.

### BRIEF DESCRIPTION OF THE FIGURES

FIG. 1 shows the graphic presentation of hydroperoxide concentration in the liver homogenate of three animal groups.

FIG. 2 shows the human LDL emission spectrum in aerated PBS suspension.

FIG. 3 shows the peroxidation kinetic curves of linoleic acid catalyzed by soy lipo-oxygenase (SLO).

FIG. 4 shows a bar graph of the extension (Ext) and initial velocity (V₀) of the SLO-catalyzed linoleic acid (LA) peroxidation reaction.

FIG. 5A shows the histological characteristics of hepatic tissue of rats fed a choline-deficient diet, Control group.

FIG. 5B shows the histological characteristics of the hepatic tissue of SNAC-treated rats fed a choline-deficient diet. FIG. 6A shows the histological characteristics of the liver tissue of ob/ob mice fed a methionine/choline-deficient diet (Group MCD).

FIG. 6B shows the histological characteristics of the liver tissue of ob/ob mice fed a hyperlipidemic diet (Group H)

FIG. 6C shows the histological characteristics of the liver tissue of ob/ob mice fed a MCD diet concomitantly with 30-day SNAC administration.

FIG. 6D shows the histological characteristics of the liver tissue of ob/ob mice fed an H diet concomitantly with 30-day

SNAC administration.

FIG. 6E shows the histological characteristics of the liver tissue of ob/ob mice fed a MCD diet for 30 days, with SNAC administration started at the 31st and maintained up to the 60th day.

FIG. 6F shows the histological characteristics of the liver tissue of ob/ob mice fed an H diet for 30 days, with SNAC administration started at the 31st and maintained up to the 60th day.

FIG. 7 shows the mass percent variation in animals fed control diet (C), methionine/choline-deficient diet (MCD) and hyperlipidemic diet (H), which either received or not SNAC by gavage during four weeks. Data are expressed as means±standard deviation.

### DETAILED DESCRIPTION OF THE INVENTION

The present invention refers to the development of pharmaceutical composites that comprise in their active principle a pharmaceutically effective amount of the equimolar mixture of a nitrosable thiol and sodium nitrite, where the pharmaceutically effective equimolar amounts of both components may range from 4 μmol to 40 mmol, with a preferential value of 0.6 mmol, for addition to a final mixture volume of approximately 300 mL. These substances may or may not be dissolved in water.

When such substances, preferably in the above-mentioned amounts, are conjunctly dissolved in water, in a final volume of approximately 300 mL, the active principle of the referred composites is generate, as follows: the S-nitrosothiols, which may be obtained in concentrations ranging from 13 μmolar to 130 mmolar, with a preferential value of 2 mmolar. For the present invention, the use of final volumes other than 300 mL for the mixture of S-nitrosothiols is allowed, provided they present proportional equimolar amounts of both components. The pharmaceutical composites of the present invention are effective in the treatment and/or prevention of the Fatty Liver Diseases, more specifically the Nonalcoholic Fatty Liver Disease (NAFLD).

In addition, the referred composites comprise flavoring substances; edulcorants, such as sucrose, aspartame and others; colorants; effervescent substances, such as excipients; and/or any other pharmaceutically acceptable vehicle that does not interfere with the action of the active principle of the referred composites.

The nitrosable thiol used in the present invention may be selected among glutathione, the N-acetylcystein, the cysteine, the homocysteine, the penicillamine, the captopril, the pantothenic acid derivatives, the proteins, such as albumin and hemoglobin, the thiols covalently bound to lipophilic carbon chains, and the dithiols.

The substances nitrosable thiol and sodium nitrite may also be replaced by a pharmaceutically acceptable amount of a corresponding S-nitrosothiol where the pharmaceutically effective amount of the S-nitrosothiol may range from 4 μmol to 40 mmol, with a preferential value of 0.6 mmol, for addition to a final solution volume of approximately 300 mL, thus producing S-nitrosothiol solutions with concentrations ranging from 13 μmolar to 130 mmolar, with a preferential value of 2 mmolar. For the present invention, the use of final volumes other than 300 mL for the S-nitrosothiol solutions is allowed, provided they present proportional molar amounts of S-nitrosothiols to obtain the above-mentioned concentrations. Therefore, the S-nitrosothiols used as the active principle in the present invention may be selected among S-nitrosoglutathione, the S-nitroso-N-acetylcystein, the S-nitrosocysteine, the S-nitrosohomocysteine, the S-nitrosopenicillamine, the S-nitrosocaptopril, the S-nitrosopantothenic acid derivatives, the S-nitrosoproteins like S-nitrosoalbumin and S-nitrosohemoglobin, the S-nitrosothiols covalently bound to lipophilic carbon chains, and the S-nitrosodithiols.

In addition to the S-nitrosothiols, the formulations of the present invention may contain other nitric oxide donors, such as diazeniumdiolates and nonoates.

The active principle of the referred pharmaceutical composites is preferably packaged in envelopes. The basic substances of the active principle, i.e., the nitrosable thiol and sodium nitrite, may be enclosed together in a single envelope or one envelope may contain one of the basic substances of the active principle, for example, the nitrosable thiol, and the second envelope contains the other substance of the active principle, sodium nitrite. The contents of the envelopes should be conjunctly dissolved in water in such a way that the active principle of the pharmaceutical composites of the present invention may be generated.

The substances that comprise the active principle of the pharmaceutical composites of the present invention may be presented as powders, powders diluted or dispersed in inert vehicles, water-soluble powders, granules, pastilles, pills, capsules and dragées.

Nevertheless, other forms of presentation of the basic substances of the active principle of the referred composites are also acceptable, such as simple solutions, composite solutions, syrups, elixirs, dispersions, emulsions and suspensions, or as multiple-unit dosage forms either containing excipients or not.

When such solutions are presented as solids or liquids, the pharmaceutical composites of the present invention may be packaged in any other type of receptacle, such as flasks, in a way that the pharmaceutical composites are protected from water sorption or loss and biological contamination during the storage time.

In case the basic substances of the active principle of the composites of the present invention are packaged in different envelopes or receptacles, the envelope or receptacle that contains the nitrosable thiol may additionally contain flavoring substances; edulcorants, such as sucrose, aspartame and others; colorants; effervescent substances, such as excipients; and/or any other pharmaceutically acceptable vehicle that does not interfere with the action of active principle of the referred composites.

In addition to the active principle of the pharmaceutical composites based on the S-nitrosation of a thiol by sodium nitrite, the basic substances of the active principle of the referred composites may be associated with other drugs that present a beneficial effect on the treatment of NADFL, hepatic steatosis and other diseases associated with the metabolic syndrome, such as obesity, type 2 diabetes mellitus, dyslipidemia, visceral adiposity and hypertension, including tocopherol or tocopherol acetate, or vitamin E, metformin, troglitazone, rosiglitazone, pioglitazone, clofibrate, gemfibrozil, atorvastatin and other statins, betaine and nicotinamide.

Yet, the basic substances of the active principle of the referred composites may be associated with other drugs that potentialize the effect of the S-nitrosothiols, such as the phosphodiesterase-5 inhibitors, among which sildenafil, tandalafil, vardenafil and others.

The pharmaceutical composites subject of the present invention are administrated to the patient by the oral or rectal route and are presented preferably as multiple-unit pharmaceutical forms in solid dosages, such as capsules, pills and dragées or pastilles. More specifically, each multiple unit comprehends:
at least an inert nucleus with an outer surface;
a first layer containing the S-nitrosothiol, which covers at least a first portion of the outer surface of the nucleus;
a second layer that controls the speed of S-nitrosothiol release from the first layer, which covers at least a second portion of the outer surface of the first layer; and
at least an additional coating layer.

The inert nucleus of the referred capsules additionally presents one or more sugars, such as glucose, manitol, lactose, xylitol, dextrose, sucrose among others; microcrystalline cellulose, silicon dioxide, silica, polystyrene, hydroxypropyl methylcellulose or other biocompatible polymers. In addition, the referred nucleus includes at least one of the following components: an insoluble material, such as cellulose acetate or paraffin; a soluble material, such as polyvinyl alcohol or polyethylene glycol; or a swellable material, such as hydroxypropyl cellulose.

Both the first and the second nucleus' coating layer comprise one or more S-nitrosothiol release-controlling polymers, which are selected from the following: ethylcellulose, hydroxypropyl methylcellulose, hydroxyethylcellulose, hydroxypropyl methyl phthalate, cellulose acetate, cellulose acetate phthalate, or any polymer resulting from the mixture of these components, in addition to one or more enteric polymers, which are selected from the following: cellulose acetate phthalate, cellulose acetate, hydroxypropyl methylcellulose acetate phthalate, poly(vinyl acetate phthalate), hydroxypropyl phthalate, hydroxypropyl methylcellulose phthalate, hydroxypropyl methylcellulose acetate succinate, methacrylic acid copolymers, or any mixtures of these compounds, or any polymer resulting from the mixture of these components.

Additionally, the multiple units are provided with one or more pharmaceutically acceptable excipients, which include surfactants, binders, diluents, disintegrators, lubricants, gliding agents, plastifying agents, stabilizers and colorants. These excipients are directly associated with one or more than one multiple-unit components, as follows: nucleus, first coating layer or second coating layer.

Preferably, the additional coating layers comprehend at least 3 (three) layers, as follows:
a first layer located between the nucleus and the first coating layer, which covers the first portion of the outer surface of the nucleus;
a second layer, which covers at least part of the first layer;
a third layer, which is located over the second layer and covers at least part of the second layer, where one or more additional layers include one or more sealing layers.

The components of the sealing layer are preferably selected from the following: hydroxypropyl methylcellulose, polyvinylpyrrolidone and methacrylic acid copolymers.

Additionally, the present invention refers to a method of treatment and/or prevention of the Fatty Liver Diseases, more specifically, the Nonalcoholic Fatty Liver Disease (NAFLD) by the oral or rectal administration of the composites described in the present application. The administrated amount should range from 4 μmol to 40 mmol, with a preferential value of 0.6 mmol, on a daily basis.

The period of treatment may range from approximately 1 month to chronic use. Nevertheless, the administration of the referred composites should be periodically reviewed and should be adjusted according the evolution of the clinical condition.

For better understanding of the present invention, a detailed description will be featured below in the form of examples. Nevertheless, the examples hereby described have a merely illustrative nature, not being restrictive forms of the present invention.

### Example 1

Wistar rats with hepatic steatosis induced by a choline-deficient diet were assigned to three groups to perform the tests with the administration of S-nitroso-N-acetylcysteine (SNAC), which is one of the active principles of the present invention, and to evaluate the concentration of hydroperoxides (LOOH) in the liver.

A first group of animals, group 1, was treated with a solution of S-nitroso-N-acetylcysteine, which was administrated orally to the animals at a dose of 1.4 mg/kg/day. A second group of animals, group 2, was treated with N-acetylcysteine (NAC), which was also administrated orally to the animals at a dose of 7.0 mg/kg/day. A third group of animals, group 3, did not receive any type of treatment and served as a control group during the test.

The test of the present Example showed that the treatment of group 1, to which the composite of the present invention was administered, resulted in the prevention of hydroperoxide concentration increase in the liver, compared to group 3 (control). For the animals in group 1, hydroperoxide concentration was 0.3±0.1 nmol/g protein, while for the animals in group 3 (control group), hydroperoxide concentration was 3.2±0.4 nmol/g protein.

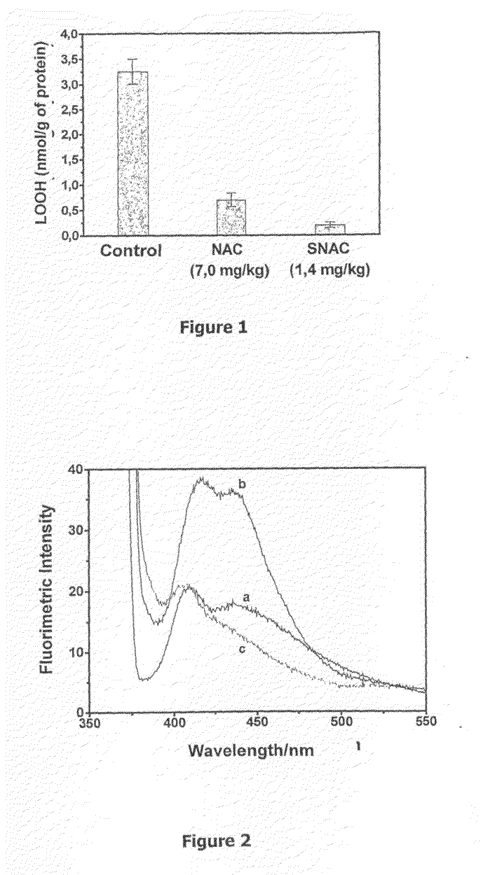

Comparing the results obtained in group 2 to those obtained in group 3 (control group), it is also observed a prevention of hydroperoxide concentration increase in the liver in group 2. For the animals in group 2, hydroperoxide concentration was 0.7±0.2 nmol/g protein, while for the animals in group 3 (control group), hydroperoxide concentration was 3.2±0.4 nmol/g protein. The results obtained in the test of the present Example and depicted graphically in FIG. 1 demonstrate that SNAC administration is much more effective in the prevention of lipid peroxidation than NAC administration, which exerts only an antioxidant effect. The greater efficiency of SNAC is associated with the biochemical activities of the nitric oxide present in this molecule.

### Example 2

For this example, approximately 15 mL of a human low density lipoprotein (LDL) suspension at a concentration of around 200 μg/mL, in phosphate buffer, pH 7.4, aerated PBS (in vitro) were previously prepared and initially divided into three parts. A first part (a) with approximately 5 mL consisting of the previously prepared suspension solely; a second part (b) with approximately 5 mL, consisting of the LDL suspension incubated in a CuCl₂ solution at a preferential concentration of 300 μmol/L for a period of around 15 hours; and a third part (c) with approximately 5 mL, consisting of the LDL suspension incubated in a CuCl₂ solution at a preferential concentration of 300 μmol/L for a period of around 15 hours, in the presence of SNAC at a preferential concentration of 300 μmol/L.

A sample of each part was submitted to fluorescence spectrophotometry. FIG. 2 features the emission spectrum for the analyzed samples. It is observed on FIG. 2 that the curve (a) presents two emission peaks at 410 and 440 nm, which may be attributed to the partial oxidation of the freshly prepared LDL suspension. The 410- and 440-nm emission peaks in the LDL suspension fluorescence spectrum correspond to the formation of a Schiff base adduct between the lipid oxidation products contained in the LDL particles (mainly the malondialdehyde [MDA]) and the free amino groups on LDL apolipoprotein (mainly apo-B-100), and are known as LDL oxidation markers.

It may be observed that both peaks increase after LDL incubation with CuCl₂, as shows curve (b). The referred peaks reflect the oxidation of the LDL suspension catalyzed by the Cu(II) ions. However, the curve (c) (incubation of the LDL suspension with CuCl₂ in the presence of SNAC) indicates the blockage of the increase of 410 and 440 nm peaks, which demonstrates SNAC's inhibitory action on LDL peroxidation. This fact is consistent with the data obtained in the in vivo tests performed in Example 1 and exhibited on FIG. 1.

### Example 3

The Example 3 shows the effect of the SNAC composite on the kinetics of linoleic acid (LA) oxidation at a preferential concentration of 18.76 μmol/L catalyzed by soy lipoxigenase (SLO). This referred effect may be shown by the analysis of two kinetic parameters: initial velocity and extension of the peroxidation reaction until the chemical equilibrium is reached.

Such parameters were analyzed by ultraviolet spectrophotometry and the kinetic curves (a′, b′, c′ and d′) were obtained from absorbance variation monitored though the band with peak at 234 nm at a preferential temperature of 37° C. This band value is characteristic for combined dienes and may, therefore, act as LA peroxidation marker.

For this Example, the first curve (a′) refers to a sample of linoleic acid whose peroxidation was catalyzed by incubation with soy lipo-oxygenase at a preferential concentration of 0.056 μmol/L; the second curve (b′) refers to a sample of linoleic acid whose peroxidation was catalyzed by incubation of the linoleic acid with a mixture of soy lipo-oxygenase in the presence of NAC at a preferential concentration of 560 μmol/L; the third curve (c′) refers to a sample of linoleic acid whose peroxidation was catalyzed by incubation of soy Lipo-oxygenase in the presence of SNAC at a preferential concentration of 56 μmol/L; and the fourth curve (d′) refers to a sample of linoleic acid whose peroxidation was catalyzed by incubation of soy lipo-oxygenase in the presence of SNAC at a preferential concentration of 560 μmol/L.

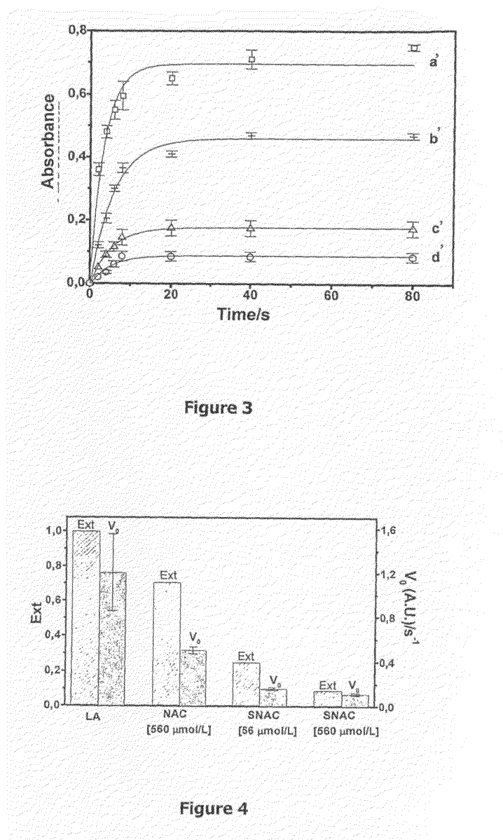

FIG. 3 illustrates the absorbance curves obtained for the groups of the present Example. The interpretation of the curves is given by the initial velocity, which corresponds to the inclination of the initial section of the curves within approximately 10 seconds, and the extension of the reaction, which corresponds to the absorbance values on the plateaus of the curves.

It may be observed that both parameters correspond to maximum when LA is incubated only with SLO, as shows curve 3(a′). Curve 3(b′) shows that, the incubation with NAC reduced the extension and velocity of oxidation, but such reduction is more accentuated in the co-incubation with SNAC, even in a concentration tenfold weaker than that of NAC (Group C), as shows Curve 3(c′). This reduction is considerably much pronounced in Group D, as shows Curve 3(d′).

These parameters are illustrated on the graph bar of the extension (Ext) and initial velocity (V₀) of the SLO-mediated linoleic acid (LA) peroxidation reaction, depicted on FIG. 4.

This figure shows that both the velocity and the extension of the reaction were significantly reduced in the presence of SNAC, even if compared to NAC in a tenfold stronger concentration. This result also demonstrates the greater blocking potential of SNAC lipid peroxidation relative to a standard antioxidant, such as NAC.

The results obtained during the Examples performed to assess the efficacy of the pharmaceutical composites subject of the present invention demonstrated a strong inhibitor effect of SNAC on the fat deposits in the hepatic tissue, which is the first step in NADLF onset.

The protective effect of SNAC observed in the present Examples may be analyzed in relation to the oxidative stress in NAFLD pathology, as previously described in the literature.

In the present Examples, it was observed that the oral administration of the SNAC composition reduced the formation of hydroperoxides (LOOH) in the hepatic tissue, which indicates that SNAC acts as a powerful inhibitor effect of lipid/lipoprotein peroxidation.

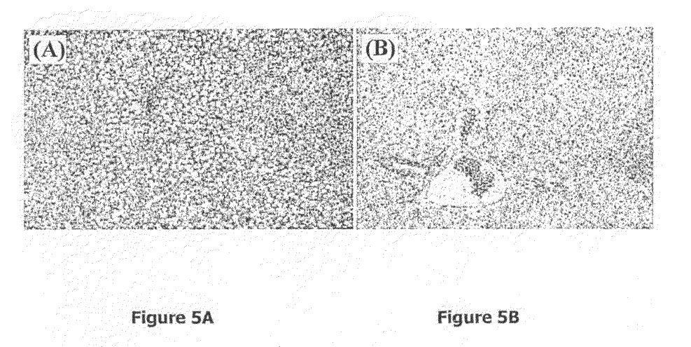

Rats fed a choline-deficient diet were selected to compose the Control Group when compared to SNAC-treated rats. Hematoxylin and eosin-stained histological sections of hepatic tissue were examined under optical microscopy, as shown on FIGS. 5A and 5B.

FIG. 5A illustrates the results obtained for the Control Group, showing moderate macrovacuolar and microvacuolar steatosis in the periportal zone. FIG. 5B shows the results obtained in the Group of SNAC-treated animals, indicating a normal liver in the periportal zone. Based on the results obtained by optical microscopy, it is evident the SNAC composite's inhibitor effect on lipid/lipoprotein peroxidation.

### Example 4

For the Example 4, ob/ob mice were selected. A first group of mice was fed a methionine/choline-deficient diet (Group MCD) and a second group of mice was fed a hyperlipidemic diet (Group H).

Animals were selected from both groups and hematoxylin and eosin-stained histological sections of the hepatic tissue of these animals were obtained and examined under optical microscopy.

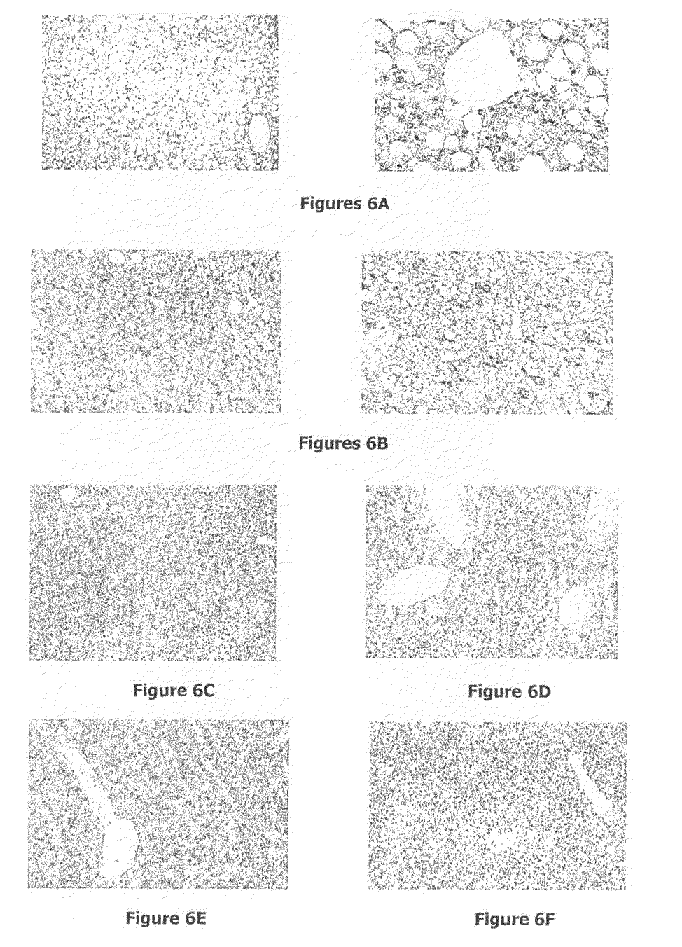

The histological characteristics of the livers of mice selected from the Group MCD revealed diffused moderate macrovacuolar and microvacuolar steatosis and inflammatory infiltrate, as shown on FIG. 6A, whereas the histological analysis of the livers of mice selected from the Group H revealed diffuse microvacuolar steatosis and discrete inflammatory infiltrate, as shown on FIG. 6B.

Other animals selected from both groups of mice, Group MCD and Group H, were submitted to a 30-day treatment consisting of SNAC administration. After the 30th day, hematoxylin and eosin-stained histological sections of the hepatic tissue of these animals were obtained and examined under optical microscopy.

FIGS. 6C and 6D exhibit the histological characteristics of the livers of mice from both groups, Group MCD and Group H, respectively. These histological characteristics were consistent with normal livers for both groups of mice submitted to their respective experimental diets.

The remaining animals in Groups H and MCD were maintained on these diets up the 60th day and were put on SNAC administration from the 31st day on. After the 60th day of treatment, hematoxylin and eosin-stained histological sections of the hepatic tissue of these animals were obtained and examined under optical microscopy.

FIGS. 6E and 6F exhibit the histological characteristics of the livers of mice from both groups, Group MCD and Group H, respectively. The histological characteristics observed were consistent with those of normal livers.

The results obtained in the tests performed in the Examples of the present invention show that SNAC administration may either block or revert NAFLD in these animal models, and that, therefore, nitric oxide donors, such as SNAC and others, have potential to be used in the treatment of other diseases associated with the metabolic syndrome, such as obesity, type 2 diabetes mellitus, dyslipidemia, visceral adiposity and hypertension.

The report of the present invention was hereby featured with descriptive and illustrative purposes, and does not intend to restrict the invention to the form depicted in this document. Therefore, variations and modifications consistent with the above-discussed issues as well as the skills or knowledge relevant to technique are within the scope of this invention.

Another finding observed in the results of the tests described in the Examples of the present invention refers to the fact that while the animals fed the Control diet presented 13% body mass increase, the animals that received the MCD diet showed little body mass increase, and those that were fed a hyperlipidemic diet (H) had their body mass increased by 28%. The treatment with SNAC practically eliminated the body mass increase in the group MCD and led to a remarkable reduction of body mass increase in the group H, after four weeks of treatment. These results demonstrate that the administration of S-nitrosothiols, such as SNAC, by the oral route has potential for the control of obesity and may lead to a reduction of body mass.

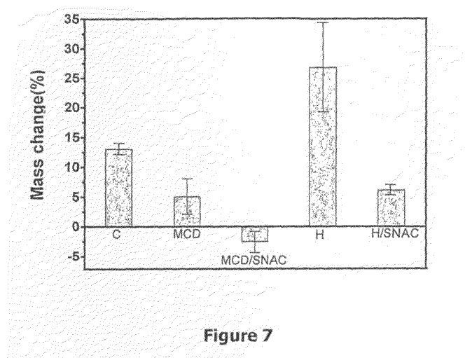

More specifically, these results show the great potential of the oral treatment with nitric oxide donors for the control of fat accumulation in the tissues and therefore for the control of obesity. This potential is emphasized by the result presented on FIG. 7, which shows that the treatment with SNAC practically eliminated the mass increase in the group of animals fed a methionine/choline-deficient diet (MCD) and led to a remarkable reduction of body mass increase in the group of animals fed a hyperlipidemic diet (H), after four weeks of treatment.

The above described modalities are intended to further outline the known ways of actualizing this invention as well as to allow that professionals in the field use the present invention in such or other modalities and with the modifications required by its specific applications or uses. It is intended that the present invention encompasses all of its modifications and variations within the scope described in this report and in the attached claims.

1.-31. (canceled)

32. Pharmaceutical composition with nitrosating action capable of modifying the action of enzymes involved in the synthesis and transport of lipids in the liver characterized by comprising in their active principle a pharmaceutically effective amount of the equimolar mixture of a nitrosable thiol and sodium nitrite, conjunctly dissolved in water, thus generating the S-nitrosothiols, which are effective in the treatment and/or prevention of the Fatty Liver Diseases and other diseases associated with the metabolic syndrome.

33. Pharmaceutical composition according to claim 32, characterized by the fact that pharmaceutically effective equimolar amounts of both basic substances range from 4 μmol to 40 mmol, with a preferential value of 0.6 mmol, for addition to a preferential final solution volume of approximately 300 mL.

34. Pharmaceutical composition according to claim 32, characterized by the fact that the S-nitrosothiols are obtained at concentrations ranging from 13 μmolar to 130 mmolar, with a preferential value of 2 mmolar.

35. Pharmaceutical composition according to claim 32, characterized by being used in the treatment and/or prevention of the Fatty Liver Diseases, more specifically the Nonalcoholic Fatty Liver Disease (NAFLD), obesity, type b 2 diabetes mellitus, dyslipidemia, visceral adiposity and hypertension.

36. Pharmaceutical composition according to claim 32, characterized by the fact that the use of final volumes other than 300 mL for the solution of S-nitrosothiols is allowed, provided they present proportional equimolar amounts of both basic substances.

37. Pharmaceutical composition according to claim 32, characterized by the fact that referred composition may additionally contain flavoring substances; edulcorants, such as sucrose, aspartame and others; colorants; effervescent substances, such as excipients; and/or any other pharmaceutically acceptable vehicle that does not interfere with the action of the active principle of the referred composition.

38. Pharmaceutical composition according to claim 32, characterized by the fact that the nitrosable thiol is selected from the glutathione, the N-acetylcystein, the cysteine, the homocysteine, the penicillamine, the captopril, the pantothenic acid derivatives, the proteins, such as albumin and hemoglobin, the thiols covalently bound to lipophilic carbon chains, and the dithiols.

39. Pharmaceutical composition according to claim 32, characterized by the fact that the nitrosable thiol and sodium nitrite may be replaced by a pharmaceutically acceptable amount of a corresponding S-nitrosothiol ranging from 4 μmol to 40 mmol, with a preferential value of 0.6 mmol, for addition to a preferential final solution volume of approximately 300 mL.

40. Pharmaceutical composition according to claim 32, characterized by the fact that the S-nitrosothiol solutions have concentrations ranging from 13 μmolar to 130 mmolar, with a preferential value of 2 mmolar.

41. Pharmaceutical composition according to claim 32, characterized by the fact that the use of final volumes other than 300 mL for the solutions of S-nitrosothiols is allowed, provided that the S-nitrosothiols are present in proportional molar amounts to obtain concentrations ranging from 13 μmolar to 130 mmolar, with a preferential value of 2 mmolar.

42. Pharmaceutical composition according to claim 32, characterized by the fact that the S-nitrosothiols used are selected from the S-nitrosoglutathione, the S-nitroso-N-acetylcystein, the S-nitrosocysteine, the S-nitrosohomocysteine, the S-nitrosopenicillamine, the S-nitrosocaptopril, the S-nitrosopantothenic acid derivatives, the S-nitrosoproteins like S-nitrosoalbumin and S-nitrosohemoglobin, the S-nitrosothiols covalently bound to lipophilic carbon chains, and the S-nitrosodithiols and other nitric oxide donors, such as diazeniumdiolates and nonoates.

43. Pharmaceutical composition according to claim 32, characterized by the fact that both basic substances may be presented as powders, powders diluted or dispersed in inert vehicles, water-soluble powders, granules, pastilles, pills, capsules and dragées, simple solutions, composite solutions, syrups, elixirs, dispersions, emulsions and suspensions, or as multiple-unit dosage forms either containing excipients or not.

44. Pharmaceutical composition according to claim 32, characterized by the fact that basic substances of the active principle are preferably packaged in envelopes.

45. Pharmaceutical composition according to claim 32, characterized by the fact that both basic substances of the active principle may be associated in a single envelope.

46. Pharmaceutical composition according to claim 32, characterized by the fact that one envelope may contain one of the basic substances of the active principle, for example, the nitrosable thiol, and the second envelope may contain the other substance of the active principle, sodium nitrite.

47. Pharmaceutical composition according to claim 32, characterized by the fact that when presented as liquids or solids, the pharmaceutical composition should be packaged in any other type of receptacle, such as flasks, which protect the basic substances of the active principle from water sorption or loss and biological contamination during the storage time.

48. Pharmaceutical composition according to claim 32, characterized by the fact that the envelope or receptacle that contains the nitrosable thiol may additionally contain flavoring substances; edulcorants, such as sucrose, aspartame and others; colorants; effervescent substances, such as excipients; and/or any other pharmaceutically acceptable vehicle that does not interfere with the action of the active principle of the referred composition.

49. Pharmaceutical composition according to claim 32, characterized by the fact that the basic substances of the active principle of the referred composition may be associated with other drugs that present a beneficial effect on the treatment of the NADFL, such as tocopherol or tocopherol acetate, or vitamin E, metformin, troglitazone, rosiglitazone, pioglitazone, clofibrate, gemfibrozil, atorvastatin and other statins, betaine and nicotinamide.

50. Pharmaceutical composition according to claim 32, characterized by the fact that the referred composition may be associated with other drugs that potentialize the effect of the S-nitrosothiols, such as the phosphodiesterase-5 inhibitors, among which sildenafil, tandalafil, valdenafil and others.

51. Pharmaceutical composition according to claim 32,
characterized by being administered to the patient by the oral route.

52. Pharmaceutical composition according to claim 32,
characterized by being administered to the patient by the rectal route.

53. Pharmaceutical composition according to claim 32, characterized as multiple-unit pharmaceutical forms in solid dosages, such as capsules, pills and dragées or pastilles, which comprehend:
at least an inert nucleus with an outer surface;
a first layer containing the S-nitrosothiol, which covers at least a first portion of the outer surface of the nucleus;
a second layer that controls the speed of S-nitrosothiol release from the first layer, which covers at least a second portion of the outer surface of the first layer; and
at least an additional coating layer.

54. Pharmaceutical composition according to claim 32, characterized by the fact that the inert nucleus additionally presents one or more sugars, such as glucose, mannitol, lactose, xylitol, dextrose, sucrose among others; microcrystalline cellulose, silicon dioxide, silica, polystyrene, hydroxypropyl methylcellulose or other biocompatible polymers, and includes at least one of the following components: an insoluble material, such as cellulose acetate or paraffin; a soluble material, such as polyvinyl alcohol or polyethylene glycol; or a swellable material, such as hydroxypropyl cellulose.

55. Pharmaceutical composition according to claim 53, characterized by the fact that both the first and the second nucleus' coating layer comprise one or more S-nitrosothiol release-controlling polymers, which are selected from the following: ethylcellulose, hydroxypropyl methylcellulose, hydroxyethylcellulose, hydroxypropyl methyl phthalate, cellulose acetate, cellulose acetate phthalate, and mixtures thereof; in addition to one or more enteric polymers, which are selected from the following: cellulose acetate phthalate, cellulose acetate, hydroxypropyl methylcellulose acetate phthalate, poly(vinyl acetate phthalate), hydroxypropyl phthalate, hydroxypropyl methylcellulose phthalate, hydroxypropyl methylcellulose acetate succinate, methacrylic acid copolymers, and mixtures thereof.

56. Pharmaceutical composition according to claim 53, characterized by the fact that additionally, the multiple units are provided with one or more pharmaceutically acceptable excipients, which include surfactants, binders, diluents, disintegrators, lubricants, gliding agents, plastifying agents, stabilizers and colorants, directly associated with one or more than one multiple-unit components.

57. Pharmaceutical composition according to claim 53, characterized by the fact that the additional coating layers comprehend at least 3 (three) layers, as follows:
a first layer located between the nucleus and the first coating layer, which covers the first portion of the outer surface of the nucleus;
a second layer, which covers at least part of the first layer;
a third layer, which is located over the second layer and covers at least part of the second layer, where one or more additional layers include one or more sealing layers.

58. Pharmaceutical composition according to claim 53, characterized by the fact that the components of the sealing layer are preferably selected from the hydroxypropyl methylcellulose, the polyvinylpyrrolidone and methacrylic acid copolymers.

59. Method of treatment of the Fatty Liver Diseases, more specifically, the Nonalcoholic Fatty Liver Disease (NAFLD) and other diseases associated with the metabolic syndrome, characterized by the fact that the composition according to claim 53 are administered to the patient by the oral route at daily doses ranging from 4 μmol to 40 mmol, with a preferential value of 0.6 mmol, preferably on a chronic use basis.

60. Method of treatment according to claim 59, characterized by the fact that the composition is administered to the patient by the rectal route at daily doses ranging from 4 μmol to 40 mmol, with a preferential value of 0.6 mmol, preferably on a chronic use basis.

61. Method of prevention of the Fatty Liver Diseases, more specifically, the Nonalcoholic Fatty Liver Disease (NAFLD) and other diseases associated with the metabolic syndrome, characterized by the fact that the composition according to claim 32 are administered to the patient by the oral route at daily doses ranging from 4 μmol to 40 mmol, with a preferential value of 0.6 mmol, preferably on a chronic use basis.

62. Method of prevention according to claim 61, characterized by the fact that the composition is administered to the patient by the rectal route at daily doses ranging from 4 μmol to 40 mmol, with a preferential value of 0.6 mmol, preferably on a chronic use basis.
